(19) United States
(12) Patent Application Publication
(10) Pub. No.: US 2009/0216132 A1
(43) Pub. Date: Aug. 27, 2009

(54) System for Continuous Blood Pressure Monitoring

(76) Inventor: Tuvi Orbach, London (GB)

Correspondence Address: THE NATH LAW GROUP, 112 South West Street, Alexandria, VA, 22314, US

(21) Appl. No.: 11/886,778

(22) PCT Filed: Mar. 21, 2006
(86) PCT No.: PCT/IL2006/000361

§ 371 Date: Dec. 30, 2008

Related U.S. Application Data
(60) Provisional application No. 60/663,259, filed on Mar. 21, 2005.

(51) Int. Cl. A61B 5/02

(52) U.S. Cl. 600/485

(57) ABSTRACT

The invention provides a system and method for monitoring blood pressure. The system includes a cuff-free non-invasive portable blood pressure monitoring device, a processor configured to process in real-time signals obtained by the portable blood pressure device to produce one or more processing products, and a portable monitor having a display displaying in real-time one or more of the processing products. In the method of the invention, a blood pressure signal is obtained from a cuff-free non-invasive portable blood pressure monitoring device. Signals obtained by the portable blood pressure device are processed in real-time to produce one or more processing products that are displayed in real-time on a display of a portable monitor. The method and system of the invention may be used in the management of hypertension.

### FIELD OF THE INVENTION

The present invention is related generally to the field of blood pressure measuring, and more particularly, to non-invasive systems for measuring blood pressure.

### BACKGROUND OF THE INVENTION

Hypertension is usually defined as persistent raised blood pressure above 140/90 mmHg. Hypertension Management (HBP Management) typically includes a combination of medication; and life style modification regarding diet, exercise, stress management and other aspects that can improve blood pressure. During this time, the pateint's blood pressure needs to be monitored. According to Guidelines published by the NICE—National Institute of Clinical Excellence in Aug. 2004, routine use of automated ambulatory blood pressure monitoring or home monitoring devices in primary care is not currently recommended because their value has not been adequately established. Therefore, in a program of hypertension management, blood pressure is monitored by having the patient periodically report to the clinic. However, blood pressure readings obtained in the clinic often do not reflect the patient's blood pressure as he carries out his daily routine. This, together with the long time intervals between feedback on the effect of the hypertension management program obtained by the blood pressure measurements, makes it difficult for the clinician to assess the effects of the program on the patient's blood pressure. It can therefore take several months to assess the effects of the program and to determined whether and how the program should be modified.

A cuff-type sphygmomanometer is a common non-invasive device used to measure blood pressure in an individual. In these devices, an inflatable cuff is wrapped around a body limb, such as an arm. The cuff is inflated to a pressure sufficient to stop blood flow in an artery which is usually detected by the cessation of sounds from the artery that occur when blood flows in the artery. As the pressure in the cuff is gradually reduced, the pressure at which a pulsatile flow in the artery is first detected is the systolic blood pressure. The pressure at which a continuous blood flow in the artery is detected is the diastolic blood pressure.

There are conditions in which it is desirable to monitor an individual's blood pressure during daily life activities and not only at the clinic. In these cases, a blood pressure measuring device is worn by the individual, that obtains and records periodic blood pressure measurements over a period of time. When the blood pressure monitoring device includes a cuff-type sphygmomanometer, the weight of the cuff must be continuously borne by the individual and the discomfort caused by the periodic inflation of the cuff and the accompanying noise interfere with the individual's ability to carry out his normal routine, especially with his ability to sleep.

U.S. Pat. No. 4,475,554 describes a non-invasive continuous blood pressure meter, comprising an inflatable flexible finger cuff which incorporates an infrared transmitter and receiver and electronic circuitry connected to the transmitter and receiver and controlling a dynamic compressor.

Another non-invasive method for measuring blood pressure is based upon a blood pulse transit time in an artery. U.S. Pat. No. 5,649,543 to Hosaka et al. discloses detecting a blood pulse in an aorta and subsequently detecting the same pulse in a peripheral artery, and calculating a blood pressure based upon the pulse transit time from the aorta to the peripheral artery. The pulse is detected in the aorta by the electrocardiograph R wave, and is detected in the peripheral artery by a photoelectric pulse wave detector.

International application having Publication No. WO0047110, incorporated herein in its entirety by reference, discloses measuring blood pressure and other parameters of cardiovascular condition using a pulse transit time. In this publication, a parameter denoted herein by κ is used where κ is defined as the ratio of the blood flow velocity to the propagation speed of the pressure pulse wave in an individual. WO0047110 discloses methods for calculating κ from ECG and pulse recordings. For example, κ may be calculated from the algebraic expression

κ=1/(1/(PEAK·v)+1),

where v is the propagation speed of the pulse wave (the pulse wave velocity) which is inversely proportional to pulse transit time (PTT), and

PEAK=k₁·PTT·PA+k₂·AREA,

where PA and AREA are respectively the amplitude and area of the pulse wave obtained from a plethysmograph signal, and k₁ and k₂ are two empirically obtained constants.

As another example disclosed in WO0047110, κ may be obtained by:

$$κ = \frac{1}{( ( \frac{1}{P A} ) + 1 )}$$

WO0047110 further discloses calculating a systolic blood pressure (SP), diastolic blood pressure (DP), Young's modulus, cardiac output (CO), vascular resistance (VR) and vascular compliance (VC) of an individual using algebraic expressions involving κ.

None of the above cited publications provide real-time feedback to the patient on his cardiovascular condition.

### SUMMARY OF THE INVENTION

### Glossary

There follows a glossary of terms used herein, some of which are standard, others having been coined, together with their abbreviations.

Plethysmograph (PG)—An instrument for measuring blood flow.

Pulse Transit Time (PTT)—The elapsed time between the arrival of a pulse pressure peak at two points in the arterial system, or the elapsed time between a particular point in the ECG signal and the arrival of the consequent pulse wave at a particular point in the arterial system.

Cardiac output (CO)—The blood volume pumped into the aorta by the heart per minute.

Vascular compliance (VCL)—The ratio of the change in the blood vessel volume to the change in pressure.

AREA—The area under the peak of a plethysmograph signal.

Peak Amplitude (PA)—The amplitude of the peak of a plethysmograph signal.

Systolic Pressure (SP)—The blood pressure during the contraction phase of the cardiac cycle.

Diastolic Pressure (DP)—The blood pressure during the relaxation period of the cardiac cycle.

Blood Pressure (BP)—The blood pressure.

Blood Pressure Monitor (BPM)—A device monitoring the blood pressure.

Heart Rate Variability (HRV)—Changes in the Heart rate that can be assessed in several ways (e.g. variance, FFT).

Hypertension or High Blood Pressure (HBP)—Hypertension means high blood pressure. This generally means:

High systolic blood pressure is consistently over 140 mm Hg

High diastolic blood pressure is consistently over 90 mm Hg—(according to Medline Plus—US National Institutes of Health)

Hypertension Management (HBP Management)—The process of managing a patient suffering from hypertension. This process may include monitoring BP, prescribing medications; educating the patient regardin.

SmartPressure—a non invasive continuous blood pressure monitor apparatus according to this invention

SmartHeart or SmartECG—the mobile Chest Smart pressure which integrates ECG sensor with Smartpressure.

The present invention provides a system and method for continuous monitoring of blood pressure of an individual during daily life activities while providing bio-feedback in real time on the individual's cardiovascular state. As explained below, the availability of information on the individual's cardiovascular state provided in real-time during daily activities both to the patient and the clinicians allows intervention in real-time in order to affect the cardiovascular state.

The system of the invention comprises a portable cuff-free blood pressure monitoring device and a portable monitor. A processor processes signals from the blood-pressure monitoring device and displays the signals or the processing products of the signals in real-time on a display associated with the monitor. The processing product may include, for example, a systolic blood pressure (SP), a diastolic blood pressure (DP), a Young's modulus, a cardiac output (CO), a vascular resistance (VR) and a vascular compliance.

In a preferred embodiment of the invention, the blood pressure monitoring device comprises a blood pulse sensor and an ECG sensor. The processor is configured to calculate one or more cardiovascular parameters in real-time from signals obtained by the blood pressure sensor and the ECG sensor.

The blood pressure calculated by the processor may be used to trigger an alert. For example, a blood pressure above or below a specified level or a rate of change of blood pressure above a specified value may trigger an alert or may cause the sensor unit to change its mode of operation, for example to start transmitting or to start storing more detailed information of the physiological parameters.

Communication between the processor and the monitor may be via a wired connection, for example, via a universal serial bus (USB). In this case, the monitor may be, for example, a laptop personal computer (LPT), a media player such as Apple IPOD®, or an electronic note-book. In a preferred embodiment, communication between the processor and the monitor is wireless. The monitor may be, for example, a cellular phone or a personal digital assistant (PDA), configured to communicate with the processor.

In a most preferred embodiment, the portable monitor is a mobile telephone and communication between the processor and the mobile telephone is via a wireless communication protocol such as “Bluetooth”. Other wireless communication protocols that may be used include, for example, RF bidirectional wireless communication, infra-red (IR) communication, and ultrasonic communication. Specific programs necessary for interfacing with the processor for displaying the results of the processing on the display and providing feedback to the individual may be uploaded onto the cellular phone wirelessly in the same way that a new game or ring tone is up loaded to a cellular phone.

When the monitor is a mobile phone, it may transmit data received from the processor to a remote server where in-depth analysis of the data may be performed. The remote server may provide, for example, additional processing of the data obtained by the blood pressure monitor, and feedback including recommendations to the individual. The server may further issue an alert to the individual of his condition, or summons a rescue team to assist the individual when an emergency situation has been detected. The remote server may be linked to a viewing station where a human expert can study and interpret the data, and transmit to the mobile phone recommendations to the individual.

The blood pressure monitoring device is preferably adapted to be worn by the individual so as to allow continuous blood pressure monitoring. For example, the blood pressure monitoring device may be adapted to by worn on the individuals chest, wrist or finger.

In a preferred embodiment of the invention, the blood pressure monitoring device comprises a blood pulse sensor and an ECG sensor and the processor is configured to calculate one or more cardiovascular parameters in real-time from signals obtained by the blood pressure sensor and the ECG sensor by the method disclosed in WO0047110, cited above. A calibration process is first carried out in order to obtain a value of the parameter κ of the individual that is subsequently used by the processor to calculated one or more cardiovascular parameters of the individual as disclosed in WO0047110.

Thus, in its first aspect, the present invention provides a system for monitoring blood pressure in an individual comprising:
(a) a cuff-free non-invasive portable blood pressure monitoring device;
(b) a processor processing in real-time signals obtained by the portable blood pressure device to produce one or more processing products; and
(c) a portable monitor having a display displaying in real-time one or more of the processing products.

In its second aspect, the invention provides a method for monitoring blood pressure in an individual comprising:
(a) Obtaining a blood pressure signal from a cuff-free non-invasive portable blood pressure monitoring device;
(b) processing in real-time signals obtained by the portable blood pressure device to produce one or more processing products; and
(c) displaying in real-time one or more of the processing products on a display of a portable monitor.

In its third aspect, the invention provides a method for managing hypertension in an individual comprising:
(a) periodically measuring blood pressure using a cuff-free blood pressure system comprising:
   (i) a cuff-free non-invasive portable blood pressure monitoring device;
   (ii) a processor processing in real-time signals obtained by the portable blood pressure device to produce one or more processing products; and
   (iii) a portable monitor having a display displaying in real-time one or more of the processing products.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for continuous blood pressure monitoring in accordance with one embodiment of the invention;

FIG. 2 shows a block diagram of a sensor unit for use in the system of FIG. 1;

FIG. 3 shows the sensor unit of FIG. 2 in the form of a chest sensor;

FIG. 4 shows the sensor of FIG. 2 in the form of a wrist sensor;

FIG. 5 shows the sensor of FIG. 2 in the form of a finger sensor;

FIG. 6 shows the sensor of FIG. 2 configured to be attached to a mobile phone;

FIG. 7 shows a method of calibrating the system of FIG. 1; and

FIG. 8 shows a method for managing hypertension in accordance with the invention.

### DETAILED DESCRIPTION OF THE INVENTION

The following detailed description is of the best presently contemplated modes of carrying out the present invention. This description is not to be taken in a limiting sense, but is made merely for the purpose of illustrating the general principles in accordance with the present invention. The scope of the present invention is best defined by the appended claims.

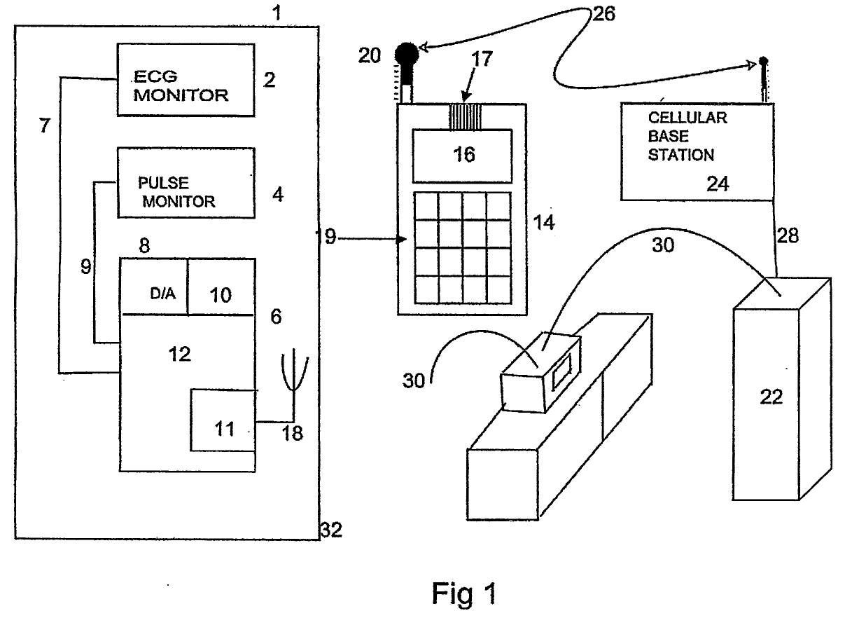

FIG. 1 shows a block diagram of a system 1 for continuously monitoring the cardiovascular state of an individual in accordance with one embodiment of the invention. The system 1 comprises a portable sensor unit 32 and a portable monitor 14. The sensor unit 32 includes an ECG sensor 2 and a pulse sensor 4. The ECG sensor 2 and the pulse sensor 4 may be included within a single unit adapted to be worn or carried by the individual, or they maybe in separate units each of which is adapted to be worn or carried by the individual. The system 1 further comprises a CPU 6. Signals 7 generated by the ECG sensor 2 indicative of the individual's ECG and signals 9 generated by the pulse sensor 4 indicative of the individual's pulse, are input to the CPU 6. The CPU 6 includes a digital to analog converter 8, a memory 10 and a processor 12. The memory 10 may be a Read Only Memory (ROM) storing a pre-installed program, a Random Access Memory (RAM), a non-volatile memory such as flash memory, or a combination of these types of memory.

After analog to digital conversion, the signals 7 and 9 are processed in real time by the processor 6. The processing may include amplification and signal conditioning filtering. The processing includes calculation of a blood pressure of the individual in a calculation involving the processing results of the signals 7 and 9. The sensing unit 32 thus functions as a cuff-free blood pressure monitor. The signals 7 and 9, and the results of the processing may be stored in the memory 10, preferably in a compressed form, for further analysis at a later time. Such a log may stored in the memory 10 may span a duration of several minutes or hours. The results of the processing are transmitted in real time from the CPU 6 to a portable monitor 14. The portable monitor 14 includes a display 16 for displaying in real time data transmitted to the monitor 14 from the CPU 6. As explained below, the monitor 14 may provide visual biofeedback to the individual by means of the display 16 and optionally audio biofeedback by means of a speaker 17.

The CPU 6 further includes a communication module 11 for communicating with the portable monitor 14. Communication between the CPU 6 and the monitor 14 may be via a wired connection, for example, via a universal serial bus (USB). In this case, the monitor 14 may be, for example, a laptop personal computer (LPT), a media player such as Apple iPOD®, or an electronic note-book. The monitor 14 may be provided with a keypad 19 which is used to control the operation of the monitor 14, the sensor unit 32, or both.

In a preferred embodiment, communication between the CPU 6 and the monitor 14 is wireless, a shown in FIG. 1. In this case the communication module 11 and the monitor 14 include antenna 18 and antenna 20, respectively. The monitor 14 may be, for example, a cellular phone (as shown in FIG. 1) or a personal digital assistant (PDA), configured to communicate with the CPU 6 and preferably equipped with any one or more of a processor configured to perform data analysis, a memory, a display, audio output, input means such as keypad, microphone and sketchpad. Transmission of data from the CPU 18 to the monitor 14 may occur upon command or may be initiated automatically, for example, when the sensor 32 is in the vicinity of the monitor 14.

In a most preferred embodiment, the portable monitor 14 is a mobile telephone and communication between the communication module 11 and the mobile telephone is via a wireless communication protocol such as “Bluetooth”. Other wireless communication protocols that may be used include, for example, radiofrequency (RF) bidirectional wireless communication, infra-red (IR) communication, and ultrasonic communication. Specific programs necessary for interfacing with the CPU 6, displaying the results of the processing on the display 16, and providing feedback to the individual may be uploaded onto the cellular phone 14. For example, a program may be loaded into a cellular phone wirelessly in the same way that a new game or ring tone is up-loaded to a cellular phone.

When the monitor 14 is a mobile phone, it may transmit data received from the CPU 6 to a remote server 22 where in-depth analysis of the data may be performed. In one embodiment, the mobile phone 14 communicates with a cellular base-station 24 via a cellular RF link 26. Alternatively, it may communicate with the server 22 using a cellular data exchange protocol, such as GPRS. The cellular base station 24 is linked to the remote server 22 by a data link 28. The remote server 22 may provide, for example, additional processing of the data obtained by the sensor unit 32, initial and updating of the mobile monitor 14 and sensor unit 32, programming, and feedback including recommendations to the individual. The server 22 may further issue an alert to the individual of his condition, or summons a rescue team to assist the individual when an emergency situation has been detected. The mobile monitor 14 may be equipped with means to establish its geographical location of the system 1 such as Global Positioning System (GPS), which may be used to direct the rescue team to the individual when he is in distress, such as during a cardiac mishap.

An additional data link 30, such as a Local Area Network (LAN) an Internet networking, an RF cellular link, or a public switched telephone network (PSTN), may be used to connect the remote server 22 to a viewing station 30 where a human expert can study the data, interpret the data, and transmit to the mobile phone 14 recommendations to the individual.

When the monitor 14 is not a mobile phone, it may communicate with the server 22 using a standard or proprietary protocol such as connection to a PTSN using a modem or an asymmetric digital subscriber line (ADSL), a local area network (LAN), or a wireless LAN (WAN), etc.

The blood pressure calculated by the processor 12 may be used to trigger an alert, to be displayed on the display 16 or to change the mode of operation of the sensor unit. For example, a blood pressure above or below a specified level or a rate of change of blood pressure above a specified value may trigger an alert or may cause the sensor unit to change its mode of operation, for example to start transmitting or to start storing more detailed information of the physiological parameters.

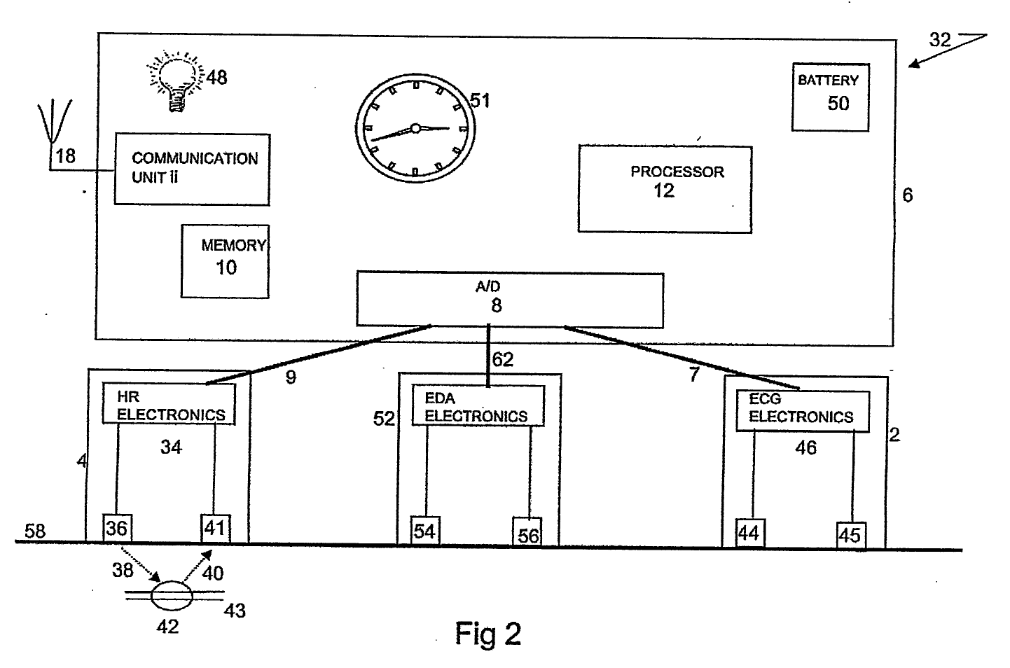

FIG. 2 shows a block diagram of the sensing unit 32 comprising the ECG monitor 2, the pulse monitor 4 and the CPU 6 in accordance with an exemplary embodiment of the invention. In the sensor unit 32, the pulse monitor 4 is a plethysmograph. The plethysmograph comprises heart rate (HR) or plethysmograph electronics 34 and a light source 36 that illuminates one or more blood vessels 43 in a tissue 42 under the individual's skin 58 with emitted light 38. Light 40 reflected from the skin 42 is received by a light detector 41. The intensity of the scattered light 40 depends on the blood flow in the blood vessels 43 under the skin 42. The signal 9 in this case generated by the plethysmograph electronics 34 is indicative of the blood volume in the blood vessels 43, and thus may be used to monitor blood flow.

Alternatively, the pulse monitor 4 may be based upon a Piezo-electric transducer (not shown). A Piezo-electric sensor is applied to the skin surface that senses expansion of one or more blood vessels due to the changing volume of blood in the vessels. Instead of being in direct contact with the skin surface, a Piezo-electric sensor may be in contact with a material or structure such as a fluid or film that transfers changes in the pressure or the shape of the organ to the Piezo-electric sensor.

In the sensing unit 32, the ECG monitor 2 includes ECG electrodes 44 and 45 connected to ECG electronics 46 in contact with the skin surface 58 when the sensing unit 32 is applied to the skin surface 58 that are used to monitor ECG signal.

In the sensing unit 32, the CPU 6 includes a battery 50 providing power for all functions of the sensing unit 32. An indicator 48 provides a sensible signal such as a visual or audio signal indicative of the status of the sensing unit such as “on/off”, “low battery” or the physiological state of the user based on the data from the sensors. A clock 51 provides a “time stamp” associating the stored data with the time of data acquisition. The sensor unit 32 may also include switches for turning the unit on and off, to change the mode of operation or commands received by the communication (not shown).

The sensor unit 32 may include additional sensors to monitor heart, breathing temperature or stress functions and provide some of the monitoring and alert functions disclosed for example in PCTIL/2006/000230. For example, the sensing unit 32 may include additional sensors such as an electro-dermal activity (EDA) 52 for monitoring the electro-dermal activity of the individual's skin surface 58 The EDA sensor comprises at least a first electrode 54 and a second electrode 56 in contact with the skin surface 58 when the sensing unit 32 is applied to the skin surface 58. EDA electronics 60 monitor the skin resistively by applying a very low electric voltage between the first and second electrodes 54 and 56, so as to create an electrical current in the skin between the electrodes. The EDA electronics 60 generates a signal 62 indicative of the skin resistively that is input to the processor 12. The EDA sensor is optional, it can be taken out or replaced by additional sensors such as respiration monitor or thermometer.

The processor 12 receives the signals 7, 9, and optionally other signals, such as the signal 62 and processes the data in real time according to instructions stored in the memory 10. The processing includes determining a blood pressure of the individual in real time so as to allow continuously monitoring of the individual's blood pressure. Any one or more of the signals 7, 9, and other signals, such as the signal 62, may be transmitted to the remote server 22 by the communication module 11, as explained above, together with the calculated blood pressures.

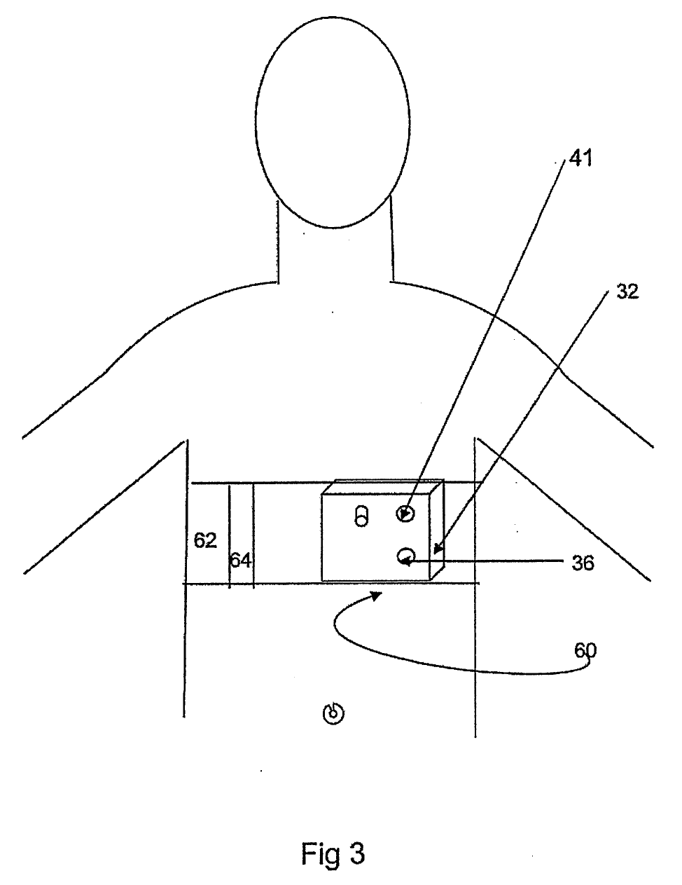

FIG. 3 shows the sensor unit 32 in the form of a chest sensor 60 adapted for being attached to the individual's chest. The chest sensor 60 may be attached to the individual's chest skin using a strap 62 wrapped around the individual's trunk and secured with a buckle 64. Alternatively, the chest sensor 60 may be affixed to the chest using an adhesive, for example the adhesive of the ECG electrodes, or hung as a pennant around the neck and operated when applied to the chest as required. As yet another alternative, the chest sensor 60 may be stored away, for example in the individual's pocket and applied to the chest when required. When the chest sensor is applied to the individual's chest skin, at least one of the ECG electrodes 44 and 45 is applied to the chest skin. The other ECG electrode in the chest sensor may either be applied to the chest skin to obtain ECG signals from the chest. Alternatively, the other ECG electrode may be on the side facing away from the chest, in which case, an ECG signal is obtained by the individual applying a finger or a part of his arm to this electrode.

The chest sensor unit is configured so that the light source 36 and the light detector 41 of the plethysmograph 4 are exposed on the surface of the chest sensor 60 away from the chest when the chest sensor 60 is applied to the chest skin. The individual presses a finger to provide a reading of the blood flow in this part to the plethysmograph. The chest sensor may be provided with an on off switch 64 that is depressed when a finger or other body part is applied to the chest sensor in order to activate the sensor thus saving battery power.

In another variation of the chest sensor 60, instead of integrating the light source 36 and the light detector 41 of the plethysmograph in the chest unit 60, a separated pulse sensor may be attached to the finger or earlobe with either wired communication to the chest sensor or wireless communication such as Bluetooth to the monitor 14. In this case, the chest unit 60 also incorporates a communication module such as Bluetooth to communicate with the monitor 14.

The chest unit can monitor continuously the individual's ECG and heart rate, and only when the user touches the pulse sensor, is a pulse transit time (PPT), and any other parameter values, calculated.

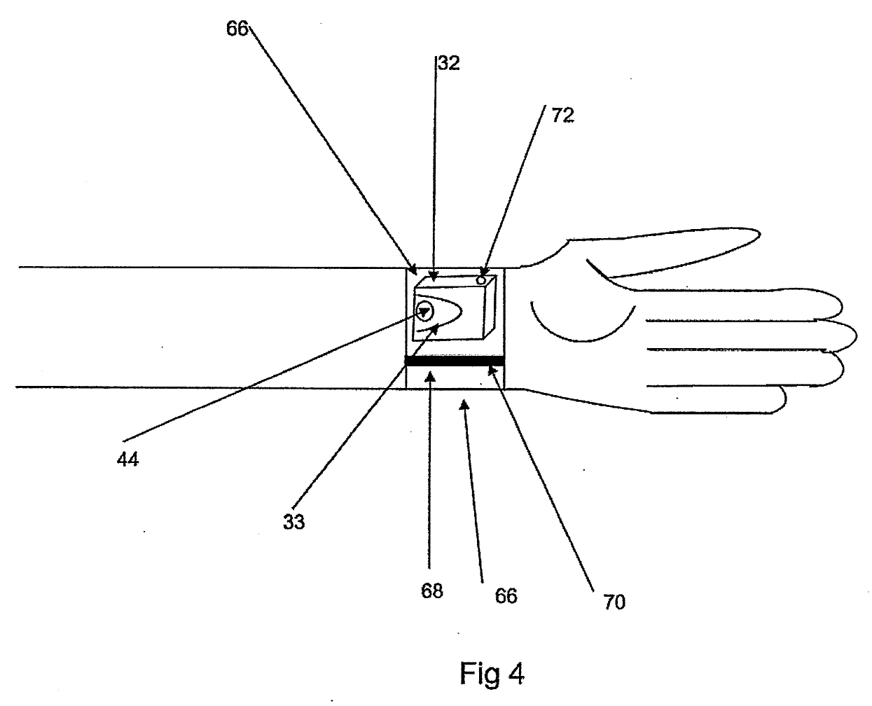

FIG. 4 shows the sensor unit 32 in the form of a wrist sensor 66 adapted for being attached to the individual's wrist. The wrist sensor 66 is attached to the individuals a wrist with a strap 68 secured with a buckle 70. The wrist sensor 66 is preferably constructed in a shape and size similar to that of a wristwatch and may optionally include functions of a watch such as displaying the time and date, acting as an alarm clock and storing data such as phone numbers etc.

When the wrist sensor 66 is worn on the individual's wrist, the ECG electrode 45 is applied to the wrist skin and is not seen in the perspective of FIG. 4. The other ECG electrode 44 in the wrist sensor is on the side facing away from the wrist, in which case, an ECG signal is obtained by the individual applying a finger to the electrode 44. The wrist sensor is preferably configured with a receptacle 33 so that the electrode 44 is consistently applied to the same location on the finger.

The wrist sensor 66 is configured so that the light source 36 and the light detector 41 of the plethysmograph 4 are applied to the carpal region of the wrist (and are not visible in the perspective of FIG. 4) where the blood flow is most noticeable when the wrist sensor 60 is worn on the wrist. In a preferred embodiment of the wrist sensor, it is configured so that the user has to touch with his other hand's finger a small area of the wrist sensor which is designed to include both the plethysmograph sensors 36 and 41 and the second ECG electrode.

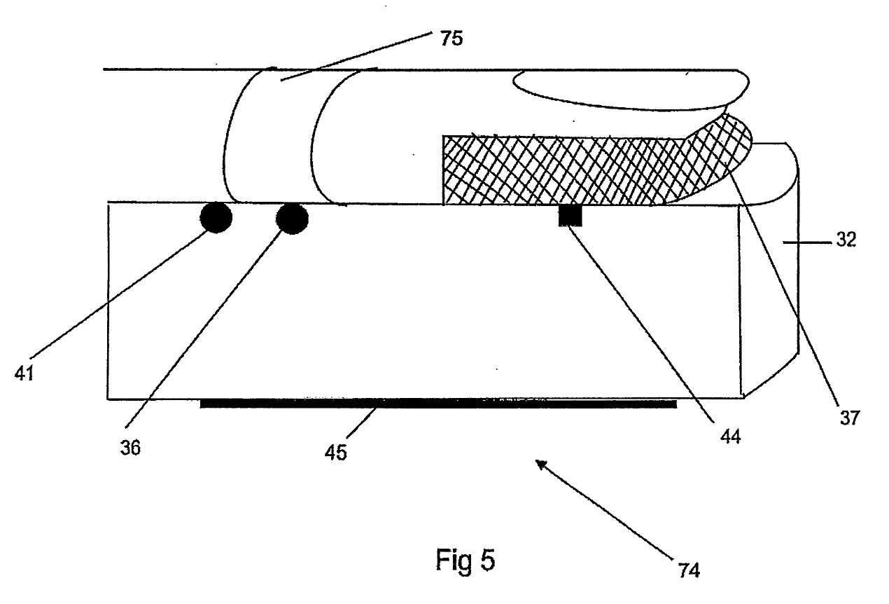

FIG. 5 shows the sensor unit 32 adapted for obtaining ECG and plethysmograph signals in the form of a finger sensor 74 adapted for being attached to the individual's finger. The finger sensor may be attached to the finger using a strap 75, or the sensor module may be shaped so that a finger may be pressed onto it. The finger sensor is preferably configured with a receptacle 37 so that the electrode 44 is consistently applied to the same location on the finger.

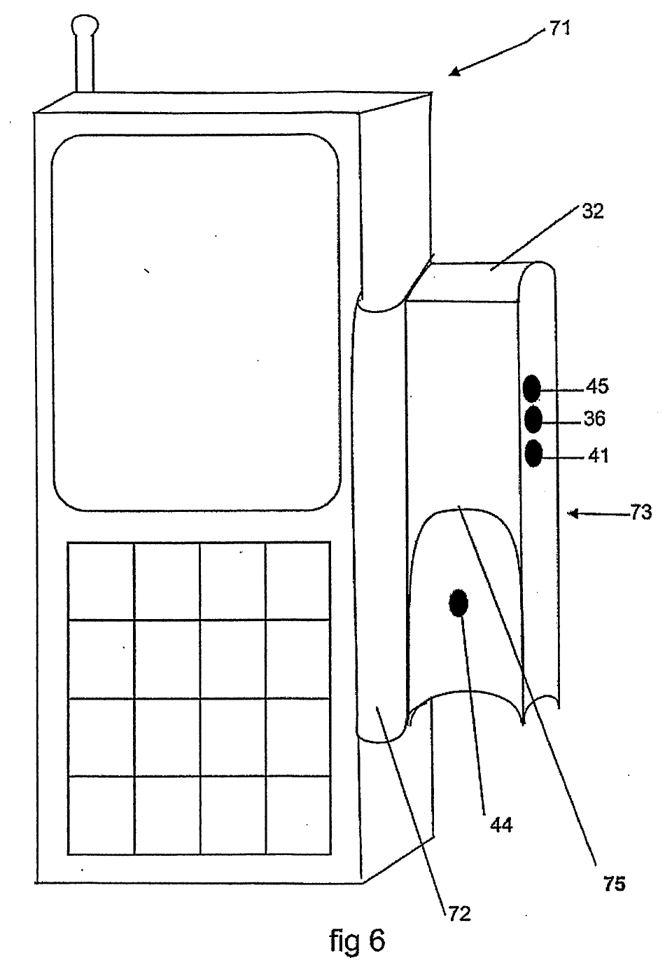

FIG. 6 shows the sensor unit 32 in the form of a sensor 73 configured to be attached to a mobile telephone 71, for example, by means of a clip 72. In this case the ECG electrode 44 is touched by one of the individual's hand while the ECG electrode 45 and the light source 36 and the light detector 41 of the pulse sensor 4 are covered by the individual's other hand. For example, the electrode 45, the light source 36 and the light detector 41 may be in contact with the individual's left hand grasping the telephone 71 while the individual touches the electrode 44 with a finger of his right hand. The sensor 73 is preferably configured with a receptacle 75 so that the electrode 44 is consistently applied to the same location on the finger.

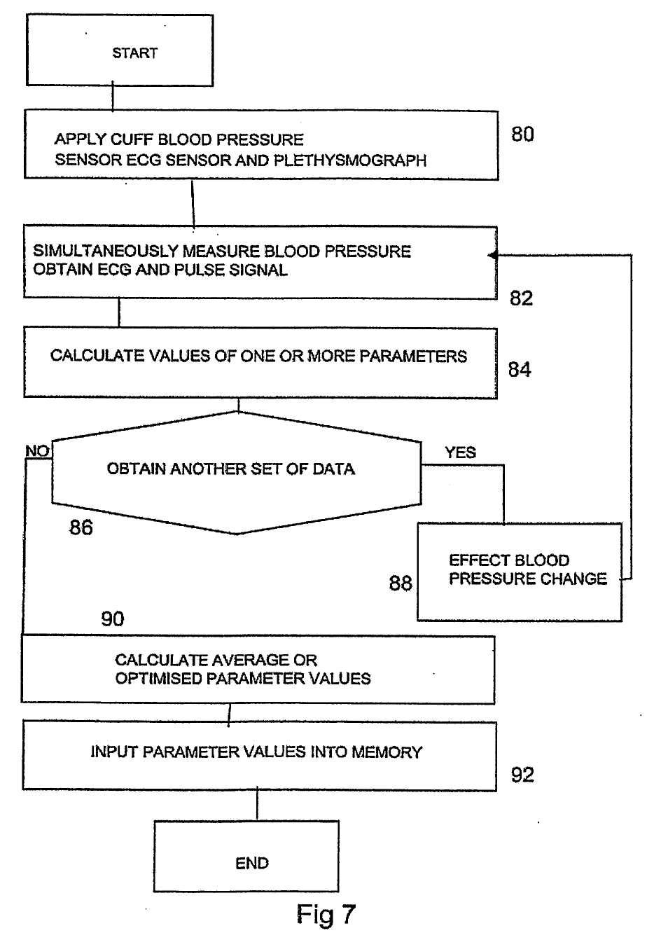

The system 1 of the invention is preferably calibrated for the individual prior to use. FIG. 7 shows a flow chart of a calibration process that may be used in accordance with the invention. In step 80, a cuff blood pressure sensor, an ECG sensor and a plethysmograph are applied to the individual. In step 82, simultaneously, the individual's blood pressure is measured using the cuff blood pressure sensor, an ECG signal is obtained using the ECG sensor and a pulse signal is obtained using the plethysmograph. In step 84, the data obtained in step 82 are used to calculate the values of one or more parameters of the individual that will be used by the processor 12 to calculate a blood pressure of the individual from ECG and plethysmograph readings obtained by the ECG sensor 2 and the pulse monitor 4, respectively. In step 86, it is determined whether another set of blood pressure, ECG and plethysmograph readings are to be obtained. If yes, the process continues to step 88 and a blood pressure change is effected in the individual and the process returns to step 82 with the values of one or more parameters of the individual being obtained under the new conditions. If at step 86 it is determined that another set of blood pressure, ECG and plethysmograph readings is not to be obtained, the process continues to step 90 with the calculation of average or optimized values of the one or more parameters. In step 92, the calculated parameters are input to the memory 10, and the calibration process ends.

Effecting a blood pressure change in the individual in step 88, may be performed, for example, by the individual changing his position or performing a physical task in order to increase his blood pressure. Alternatively or additionally the individual may rest or relax to reduce his blood pressure. A blood pressure change may also be affected by the user performing mental tasks such as meditation, doing a mental calculation, or by medications

The calibration process may be performed at a facility where a cuffed blood pressure sensor equipped to interface with the CPU 6 is located. Alternatively the cuff blood pressure sensor can be configured to interface with the monitor 14 using wireless or wired communication. The monitor 14 can transmit the blood pressure data to the sensor unit 32 using wireless or wired communication. Alternatively, the calculated parameters may be manually input to the sensor unit 32 via a key pad 33. It also should be noted that in some cases, it is sufficient to determine changes in blood pressure or a rate of change in blood pressure or in other physiological parameters while absolute calibration is not necessary.

In a preferred embodiment of the invention, the calibration process involves calculation of a parameter denoted herein by κ defined as the ratio of the blood flow velocity to the propagation speed of the pressure pulse wave in an individual. WO0047110 discloses methods for calculating κ from ECG and pulse recordings. For example, as shown in this publication, κ may be calculated from the algebraic expression

κ=1/(1/(PEAK·v)+1),

where v is the propagation speed of the pulse wave (the pulse wave velocity) which is inversely proportional to pulse transit time (PTT), and

PEAK=k₁·PTT·PA+k₂·AREA,

where PA and AREA are respectively the amplitude and area of the pulse wave obtained from a plethysmograph signal, and k₁ and k₂ are two empirically obtained constants.

As another example disclosed in WO0047110 κ may be obtained by:

$$κ = \frac{1}{( ( \frac{1}{P A} ) + 1 )}$$

Slow (0.01-0.05 Hz) fluctuations in vascular radius (vasomotor tone) can optionally be filtered out from the plethysmograph signal in order to increase the accuracy of the κ measurement. This can be carried out, for example, by replacing PEAK with PEAK/(slow component of PEAK)². The slow component of PEAK can be obtained, for example, by low-pass filtering of the pulse wave.

PTT can be obtained, for example, as the time lapse between a particular point in the ECG wave, for example the R peak, and the arrival of the corresponding pressure wave at a pulse detector such as a plethysmograph. Other means for measuring PTT comprise, for example, a pair of plethysmograph sensors that are attached to the skin along the same arterial vessel and separated from one another. In this case, the PPT is the time lapse between the arrival of a pressure wave at the two locations.

The processor 12 may be configured to calculate a Young's modulus, vascular resistance, cardiac output, and vascular compliance of the individual. As explained above, WO0047110 discloses calculating a Young's modulus, vascular resistance, cardiac output, and vascular compliance of the individual using the following algebraic expressions involving κ.

Systolic Pressure (SP)

Method 1

SP=ρv²Φ(κ,γ),

where ρ is the blood density, γ is the thermodynamic Poisson exponent of the blood, and

$$Φ = \frac{\sqrt{2 {κ ( γ - 1 )}^{2} + 4 \cdot ( γ - 1 ) + 1} - 1}{2 ( γ - 1 )}$$

Method 2

SP=(logv²)/α+2ρv²ε/3+λ,

where λ=(log(2ρR/E₀h))/α, where R is the radius of the artery, h is the thickness of the arterial wall, E₀ is Young modulus referred to zero pressure, and α is an empirically obtained constant.

Method 3

SP=(logv²/(1−εH²))/α+2ρv²κ/3+λ,

where ε is an empirically obtained constant and H is the heart rate.

Method 4

SP=[(logv²)/α+λ]/(1−κ).

Method 5

SP=[(logv²/(1−εH²))/α+λ]/(1−κ).

Diastolic Pressure (DP)

DP=SP−ρv²κ,

Young Modulus

Method 1

E=(2R/h)(SP−DP)/κ

Method 2

E=(2R/h)SP/Φ(κ,γ)

Method 3

E=(2R/h)ρ exp[(−λ+MP)α]

where MP is the mean pressure, MP=(SP+2·DP)/3, where SP or DP is obtained using an algorithmic expression involving κ.

Method 4

E=(2R/h)·ρ·exp ((−λ+SP·(1−κ))α)

Cardiac Output (CO)

CO=PEAK·{v·[1+SP/(2ρ·v²)]}²

where SP is obtained using an algorithmic expression involving κ, and the slow component of PEAK has been filtered out as described above.

Vascular Resistance (VR)

VR=(SP−DP)/CO.

where any one or more of SP, DP, and CO are obtained using an algorithmic expression involving κ.

Vascular Compliance (VC)

VC=PEAK/(SP−DP).

where any one or more of SP, and DP are obtained from a calculation involving κ. Other methods for obtaining vascular compliance from κ are also contemplated within the scope of the invention.

### The Effect of VC, VR and CO on Blood Pressure

Also disclosed in WO0047110 are methods for calculating whether a change in the blood pressure in an individual is due to a change in cardiac output or a change in vascular compliance. Since different physiological processes govern blood pressure changes of different origins and a different medical treatment is required for the same change in blood pressure when it arises from different origins, the present invention provides means for determining the appropriate treatment.

The relative contribution of CO to an observed change in SP is given by a parameter INDEX1 defined by

INDEX1=∂SP/∂CO−∂SP/∂VC

where any one or more of the parameters SP, CO, and VC are obtained from a calculation involving κ. An increase in INDEX1 over time is indicative of a change in SP primarily due to changes in cardiac output (CO). A decrease in INDEX1 over time is indicative of a changes in SP primarily due to a change in vascular compliance (VC).

The relative contribution of VR and CO to an observed change in SP is given by a parameter INDEX2 defined by

INDEX2=∂SP/∂CO−∂SP/∂VR

where any one or more of the parameters SP, CO, and VR are obtained from a calculation involving κ. An increase in INDEX2 over time is indicative of a change in SP primarily due to changes in cardiac output (CO). A decrease in INDEX2 over time is indicative of a change in SP and DP primarily due to a change in vascular resistance (VR).

### Research/Clinical Trials

The system of the invention may be used for continuous monitoring of blood pressure during clinical trials of blood pressure treatments. For example, the effect of medication on patients may be monitored as they carry out their daily routine. The invention can also provide other parameters such as changes of Cardiac output, PTT, change in vascular compliance, heart rate variability. This information can be of significance in the development of better treatments.

### Improve Diagnosis and Treatment

Today blood pressure is usually measured at the doctor's office when the patient is stressed on the one hand, and is not performing any physical task on the other hand. The blood pressure measurements obtained under these conditions may not be indicative of the individual's blood pressure as he goes about his daily routine. Obtaining a true representative record of the patient's blood pressure may increase diagnostic accuracy. The existing blood pressure monitoring systems can determine only systolic and diastolic blood pressure, but no information on cardiac output, PTT, or changes in vascular compliance. Since different physiological processes govern blood pressure changes of different origins and a different medical treatment is required for the same change in blood pressure when it arises from different origins, the present invention provides means for determining more accurate diagnostic and the appropriate treatment, The system of the present invention can also calculate from the ECG signal or the pulse signal. By monitoring the patient's heart rate variability (HRV) together with other parameters, the system can be used to diagnose earlier changes in the cardiovascular system and thus prevent complications.

### Clinics

Records of blood pressure before and after starting a dedicated regime of medication aimed at affecting patient blood pressure may enable better determination of optimal doses and timing of medication.

### Biofeedback

Biofeedback is used to train people to reduce their stress level. The monitoring system of the present invention allows training for blood pressure reduction. Conventional cuff blood pressure devices are not suitable for blood pressure biofeedback because they are not practical for continuous blood pressure monitoring, and they interfere with the blood flow. The system of the present invention enables the individual to implement blood pressure biofeedback processes and to train himself to reduce his blood pressure not only at home but as he goes about his daily routine.

Blood pressures obtained by the system may be combined with physiological parameters, for example, body temperature, to give a more complete indication of the user's state of mind and physical condition. For example, a visual, audio, or audiovisual biofeedback may be provided to a user indicative of a plurality of physiological parameters.

For example, an animation of a bird flying over a background terrain may be displayed on the display 16 wherein wing flapping is indicative of the individual's breathing rate, the bird's height above the ground is indicative of the individual's blood pressure and flying speed indicative of his heart rate. The individual may than train himself to produce slow flapping, slow and low flying of the bird. Similarly, background color may be indicative of EDA value. Music and audio can also give feedback, while specific components of the music reflect rate of the specific physiology parameters: e.g. Respiration rate can be represented by one instrument (e.g. flute), heart rate by another (e.g. drum), the level of the blood pressure by the music's volume, and so on.

### Hypertension Management—(HBP Management)

The existing procedure of managing Hypertension is not effective. Even in the United States, where the per capita investment in healthcare is one of the highest in the world, only 34% of the people suffering from hypertension are receiving adequate therapy according to the AHA (American Heart Association). But even these 34% are not actually cured but receive medication every day for the rest of their life. The doctors don't know what the blood pressure of these patients is during their daily life and when they are stressed.

“The cause of 90-95 percent of the cases of high blood pressure isn't known.” (Quotation from the AHA website). It is not known if the main cause of the high blood pressure is peripheral resistance, or cardiac output. It is not known how the medications affect the blood pressure during sleep, during physical activities or stress situations, and what the best medications are for a specific individual patient.

According to the NICE guidelines for management of hypertension, in order to decide whether a patient is hypertensive, the clinician has to ask the patient to arrive at his clinic three times at one month intervals, measure the patient's blood pressure twice during each visit and start medication only after the 3 months of examination. However, because consecutive follow up treatments are only approximately once a month, finding which medication is effective for the patient can be a lengthy process, often taking many months in a laborious and time consuming process of trial and error, fraught with loopholes. It can take several months to assess the implication of each medication, and to find the best combination for the specific patient. Many patients stop taking medication during this process because of negative side effects while they cannot feel (or see) the benefit of the medication. The clinician does not know if the patient has taken the medication, or if he has changed his life style. Even if the patient has complied with the instructions, both the clinician and the patient cannot know what the level of the patient's blood pressure is during daily activities, when he is driving or under stress.

Management of Hypertension (HBP Management) in Accordance with the Present Invention.

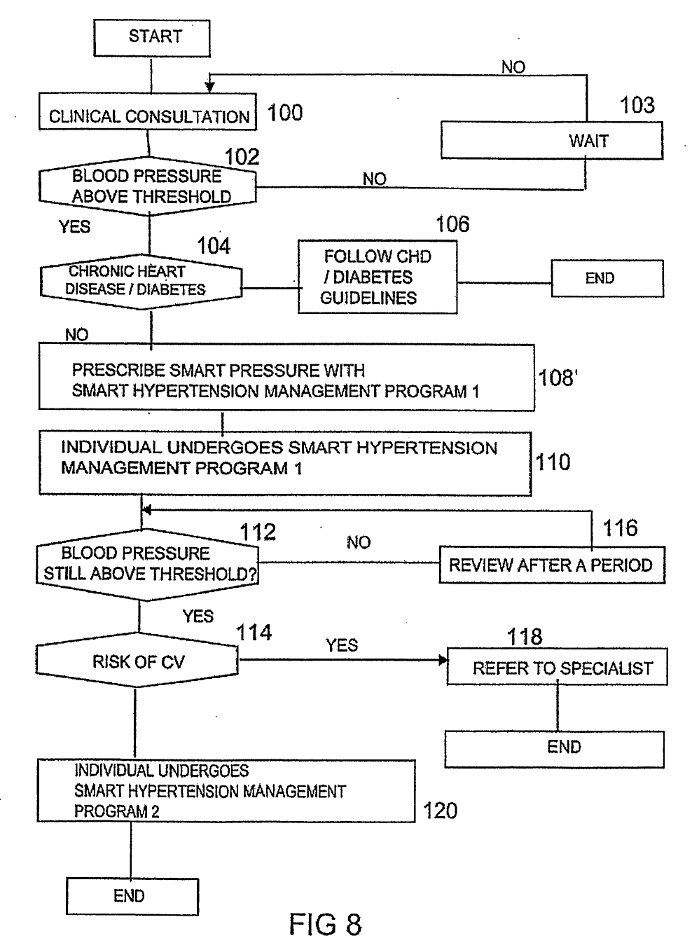

The system and method of the invention may be used in the management of hypertension. FIG. 8 shows a flow chart of a method of managing hypertension in an individual according to one embodiment of this aspect of the invention. In step 100, the individual participates in a clinical consultation that includes measuring his blood pressure. In step 102, it is determined whether the individual's blood pressure measured at the clinic is above one or more predetermined thresholds. The predetermined threshold maybe, for example, a systolic blood pressure over 140 mm Hg, and a diastolic blood pressure over 90 mm Hg. If at step 102 it is determined that the individual's blood pressure is not above the predetermined threshold or thresholds, then in step 103 the individual is instructed to wait a certain amount of time, such as twelve months, and then to return for an additional clinical consultation (step 100).

If in step 102 it was determined that the individual systolic blood pressure and/or his diastolic blood pressure is above the predetermined threshold or thresholds, then in step 104 it is determined whether he has chronic heart disease (CHD) or diabetes. If yes, then in step 106 the individual is instructed to follow CHD or diabetes guidelines, respectively, and the process terminates.

If in step 102 it is determined that the individual does not have CHD or diabetes, then in step 108 Smart Hypertension management 1 is prescribed and the individual is provided with a “SmartPressure” (a blood pressure monitoring device of the invention), and a calibration process is implemented. The individual then undergoes a “Hypertension management program I”, (step 110) in which the individual periodically monitors his blood pressure with the blood pressure monitoring device and follows the training interactive instructions. During this period the patient, his clinician and the telehealth center can check if the BP is above the recommended ranges, how it is changes during specific activities, whether changes in the individual's life style are enough to keep the BP in the recommended ranges or if medications are needed. After a predetermined time and depending on the ranges of the BP, the process continues at step 112 with the individual returning for another clinical consultation where it is determined whether the blood pressure is still above the predetermined threshold(s) and whether or not the individual still needs medication and or other examinations. This consultation can be in a clinic or using “Telehealth” using the “SmartPressure” and interactive audio or video conferencing (e.g. using a 3G smart phone). If at step 112 it is determined that the individual is not suffering from hypertension, then in step 116, the individual is instructed to review his condition, and keep healthy life style, and after a period of time to return for an additional consultation (step 112).

If in step 112 it is determined that the individual is suffers from hypertension, then in step 114 it is determined whether the individual has a cardiovascular (CV) risk. If yes, then in step 118 the individual is referred to a specialist and the process terminates. If no, then in step 120, the individual undergoes a “Hypertension management program II” which is mainly self help with telehealth support management.

The “Hypertension management program II” involves the individual, his primary caregiver and a “telehealth” centre, such as the server 22 and the viewing station 30. In the Hypertension management program II, the individual monitors his cardiovascular state as explained above with reference to the system 1. The individual may be instructed, for example, to measure his blood pressure, for example, once per day. He is also expected to comply with a lifestyle training program which may be presented to the individual as a multimedia education program. This training program sets targets, and lifestyle changes that he individual is urged to implement in his life. The training program is also designed to teach the individual how to manage stress in his life.

Data relating to the individual's cardiovascular state obtained by the individual are transmitted to the telehealth center, as described above in reference to FIG. 1. This information is also provided to the individual's primary caregiver. The telehealth center and/or the caregiver may modify the life style training program in response to data relating to the individual's cardiovascular state including, for example, blood pressure measurements transmitted to the telehealth center or the caregiver. The caregiver may also recommend modification of the medication regime of the individual.

If the hypertension management program II does not produce a satisfactory improvement in the individual's cardiovascular state, the individual might be instructed to undergo a more stringent hypertension management program (“Hypertension management program III”).

A high blood pressure reading may trigger an alert in the form of a voice message or an SMS to the mobile phone to remind the individual to take his medications, or to try and alter his lifestyle. The system may prompt the individual to perform a cuffed blood pressure measurement when an abnormal blood pressure is detected, for example a blood pressure outside a predetermined range, or when a need arises for a new calibration of the system.

The “Life Style Training Program” may be in the form of an: interactive multimedia training system that may be viewed on the display 16. The Personalized training material may include instructions for operating the system of the invention including transmission of data to the server 22 and to the viewing station 30. The training material may also instruct the individual with regard to communicating with a professional health care giver located at telehealth center (e.g. a service center including the viewing station 30), for example by internet, telephone or in a face to face meeting with the care giver. In the preferred version, the instructions can be provided to a mobile phone serving as the monitor 14, with periodic automatic reminders to the individual.

The “Life Style Training Program” includes a personalized guide with recommendations regarding all aspects of lifestyle that can help to improve blood pressure and reduce risks, e.g. diet and nutrition, exercise, stress management, behavioral and psychological advice or interactive programs such as computerized CBT (cognitive behavioral therapy). The “Life Style Training Program” may also include interactive biofeedback programs and or relaxation training.

The telehealth or service center can alert either the individual or his care giver of an abnormal situation or dispatch an ambulance to the individual. The server 22 or professional at the center system can instruct the CPU 6 to store the individual's ECG in the memory 10, to send the ECG to the server 22 or to the viewing station 30 or to instruct the individual to monitor his blood pressure, to take medication, to rest, to breath slowly, etc. The data in the centre can be used also for research, e.g. to check the effectiveness of specific medications and or life style, and or psychological or behavioral conditions and methods. In a preferred method, the monitor 14 may report to the server 22 or the viewing station 30, either automatically or by the individual, each time that he has taken medication or performed a relevant activity (e.g. using a pedometer; weighing himself). The center may also provide a “call center” providing expert advice to the individual, and may provide other information and services.

The present invention provides both the clinicians and the patients with relevant information on the patient's cardiovascular state which may be updated almost in real time. Therefore the clinician will be able to see in a short time if a medication or treatment the individual is receiving is effective, and whether he has to change the dosage, or add medication. The patient will be able to see immediately the effectiveness of the medication and or the changing of his life style on his blood pressure and will have more motivation to comply with the recommended treatment. Researchers and pharmaceutical companies will be able to monitor the effectiveness of each treatment and combinations of medication and life style on each group of patients. The Health care providers/payer—(Health plan or Health Insurance, or National Health Service) will have better information, and more important better healthcare services which may cost less.

While monitoring his blood pressure in accordance with the invention, the individual will be able to see if and how his blood pressure changes during the day, what the influence of his life style changes are on his blood pressure, and how effective is the medication. This will motivate him to comply with the caregiver's instructions. The individual will also be able to report immediately any negative side effect or other changes in his health; and either his caregiver or another professional can receive this information, give him advice or call him for consultation.

The privacy of the patient's data can be kept according to any required standard. (e.g. HIPPA in the USA); while statistical information can be accumulated for the benefit of research, and the health authorities.

This system and process can also help to accelerate the time to market of new drugs or treatments, and reduce the cost of clinical trials.

While the invention has been described with reference to certain exemplary embodiments, various modifications will be readily apparent to and may be readily accomplished by persons skilled in the art without departing from the spirit and scope of the above teachings.

It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. The terms “comprise”, “include” and their conjugates as used herein mean “include but are not necessarily limited to”.

1. A system for monitoring blood pressure in an individual comprising:
(a) a cuff-free non-invasive portable blood pressure monitoring device;
(b) a processor processing in real-time signals obtained by the portable blood pressure device to produce one or more processing products; and
(c) a portable monitor having a display displaying in real-time one or more of the processing products.

2. The system according to claim 1 wherein the blood pressure monitoring device comprises an ECG sensor and a pulse sensor.

3. The system according to claim 1 wherein one or more of the processing products is selected from the group comprising:
i. a systolic blood pressure,
ii. a diastolic blood pressure,
iii. a Young's modulus of an artery,
iv. a cardiac output,
v. a relative changes in vascular resistance, and
vi. a relative changes in vascular compliance;

4. The system according to claim 1 wherein the processing comprises calculating a ratio κ of the blood flow velocity to the propagation speed of the pressure pulse wave in an individual.

5. The system according to claim 1 wherein communication between the processor and the monitor is via a wired connection.

6. The system according to claim 5 wherein the monitor is selected from the group comprising a laptop personal computer (LPT), a media player, and an electronic note-book.

7. The system according to claim 1 wherein communication between the processor and the monitor is wireless.

8. The system according to claim 1 wherein the monitor is selected from the group comprising a cellular phone and a personal digital assistant (PDA).

9. The system according to claim 7 wherein communication between the processor and the monitor is via a protocol selected from “Bluetooth”, RF bidirectional wireless communication, an infra-red (IR) communication protocol and an ultrasonic communication protocol.

10. The system according to claim 1 wherein the blood pressure monitoring device is adapted to be worn by the individual.

11. The system according to claim 10 wherein the blood pressure monitoring device is adapted to be worn on the individual's chest, wrist or finger.

12. The system according to claim 1 further comprising an electro-dermal activity sensor.

13. The system according to claim 1 wherein the monitor is configured to communicated with a remote server.

14. The system according to claim 13 wherein the remote server is configured to analyze data received from the monitor.

15. The system according to claim 13 wherein the remote server is configured to determine recommendations for the individual based upon the data received from the monitor.

16. The system according to claim 1 wherein the monitor is configured to communicates with a viewing station.

17. The system according to claim 1 wherein the monitor is configured to generate a sensible signal when one or more of the processing products meet one or more predetermined criteria.

18. The system according to claim 17 wherein the monitor is configured to generate a sensible signal when a blood pressure measurement is above a predetermined blood pressure level.

19. The system according to claim 1 for use in managing hypertension.

20. The system according to claim 1 for use in providing biofeedback to the individual on a cardiovascular state of the individual.

21. A method for monitoring blood pressure in an individual comprising:
(a) Obtaining a blood pressure signal from a cuff-free non-invasive portable blood pressure monitoring device;
(b) processing in real-time signals obtained by the portable blood pressure device to produce one or more processing products; and
(c) displaying in real-time one or more of the processing products on a display of a portable monitor.

22. The method according to claim 19 wherein the blood pressure monitoring device comprises an ECG sensor and a pulse sensor.

23. The method according to claim 19 wherein one or more of the processing products is selected from the group comprising:
i. a systolic blood pressure,
ii. a diastolic blood pressure,
iii. a Young's modulus of an artery,
iv. a cardiac output,
v. a relative changes in vascular resistance, and
vi. a relative changes in vascular compliance;

24. The method according to claim 19 wherein the processing comprises calculating a ratio κ of the blood flow velocity to the propagation speed of the pressure pulse wave in an individual.

25. The method according to claim 19 wherein the one or more processing products are transmitted to the monitor via a wired connection.

26. The method according to claim 23 wherein the monitor is selected from the group comprising a laptop personal computer (LPT), a media player, and an electronic note-book.

27. The method according to claim 19 wherein the one or more processing products are transmitted to the monitor via a wireless connection.

28. The method according to claim 25 wherein the monitor is selected from the group comprising a cellular phone, a personal portable computer, and a personal digital assistant (PDA).

29. The method according to claim 25 wherein the one or more processing products are transmitted to the monitor via a protocol selected from “Bluetooth”, RF bidirectional wireless communication, an infra-red (IR) communication protocol and an ultrasonic communication protocol.

30. The method according to claim 19 wherein the blood pressure monitoring device is adapted to be worn by the individual.

31. The method according to claim 28 wherein the blood pressure monitoring device is adapted to be worn on the individual's chest, wrist or finger.

32. The method according to claim 19 further comprising obtaining an electro-dermal activity of the individual.

33. The method according to claim 19 further comprising transmitting data between the monitor a remote server.

34. The method according to claim 31 wherein the remote server analyzes data received from the monitor.

35. The method according to claim 31 wherein the remote determines recommendations for the individual based upon the data received from the monitor.

36. The method according to claim 19 further comprising transmitting data between the monitor a viewing station.

37. The method according to claim 19 further comprising generating a sensible signal when one or more of the processing products meet one or more predetermined criteria.

38. The method according to claim 35 comprising generating a sensible signal when a blood pressure measurement is above a predetermined blood pressure level.

39. The method according to claim 21 for use in managing hypertension.

40. The system according to claim 21 for use in providing biofeedback to the individual on a cardiovascular state of the individual.

41. A method for managing hypertension in an individual comprising:
(a) periodically measuring blood pressure using a cuff-free blood pressure system comprising:
(i) a cuff-free non-invasive portable blood pressure monitoring device;
(ii) a processor processing in real-time signals obtained by the portable blood pressure device to produce one or more processing products; and
(iii) a portable monitor having a display displaying in real-time one or more of the processing products.

42. The method according to claim 41 further comprising taking medication.

43. The method according to claim 41 further comprising providing a lifestyle training program, the lifestyle training program setting one or more targets, and lifestyle changes that the individual is urged to implement in his life.

44. The method according to claim 43 wherein the lifestyle training program trains the individual on any one or more of stress management, biofeedback, CBT, nutrition, and exercise.

45. The method according to claim 41 wherein the blood pressure is measured at least once per day.

46. The method according to claim 41 further comprising transmitting blood pressure measurements to a telehealth center.

47. The method according to claim 41 further comprising transmitting blood pressure measurements to a viewing station for viewing by a healthcare giver.
